(19) United States
(12) Patent Application Publication
(10) Pub. No.: US 2023/0040911 A1
(43) Pub. Date: Feb. 9, 2023

(54) Oil-In-Water-Type Emulsion Cosmetic

(71) Applicant: L V M H RECHERCHE, SAINT JEAN DE BRAYE (FR)

(72) Inventors: Masahiro KIKUCHI, Chiyoda-ku, Tokyo (JP); Mai OZAWA, Chiyoda-ku, Tokyo (JP)

(73) Assignee: L V M H RECHERCHE, SAINT JEAN DE BRAYE (FR)

(21) Appl. No.: 17/786,243

(22) PCT Filed: Dec. 20, 2019
(86) PCT No.: PCT/EP2019/086850

§ 371 Date: Jun. 16, 2022

(51) Int. Cl. A61K 8/06; A61K 8/73; A61Q 17/04; A61K 8/55

(52) U.S. Cl. CPC A61K 8/062; A61K 8/731; A61K 8/738; A61Q 17/04; A61K 8/55

(57) ABSTRACT

An oil-in-water-type emulsion cosmetic comprising (1) water, (2) an organic UV filter, (3) a thermo-thickening polymer and (4) an anionic surfactant, wherein component (3) is (3a) a cellulosic thermo-thickening polymer or (3b) a triblock polyoxyalky-lene-based thermo-thickening polymer, and component (4) is selected from an alkyl phosphoric acid ester of 12 to 22 carbon atoms, an amide of alkylcarboxylic acid having 12 to 22 carbon atoms and an amino acid, and a monoester of a polycarboxylic acid and a glyceride of an alkylcarboxylic acid having 12 to 22 carbon atoms.

### TECHNICAL FIELD

The present invention relates to an oil-in-water-type emulsion cosmetic.

### BACKGROUND ART

When skin is exposed to ultraviolet rays its cells undergo damage and aging of the skin is promoted. Ultraviolet rays include UV-A and UV-B, with the shorter wavelength UV-B (wavelength: 280 to 320 nm) being absorbed mainly in the surface of the skin, and also being said to produce sunburn. UV-A (wavelength: 320 to 400 nm), which has a longer wavelength than UV-B, on the other hand, reaches to the dermis of the skin and is said to produce suntan.

Sunscreen is used to protect skin from ultraviolet rays that have such effects, but since it is usually used in the high-temperature season when there is more sunlight, a problem results in that the temperature on the skin increases and fluidity of the compositions applied on the skin increases, causing it to flow off from the skin and lose its homogeneity.

Photoprotective cosmetic compositions have been proposed, in WO2008/006687, for example, whose purpose is to reduce or eliminate the effect of migration of the composition outside the application area.

### TECHNICAL PROBLEM

The photoprotective cosmetic composition disclosed in WO2008/006687 includes at least one heat-induced gelling polymer, and therefore when the temperature of the composition has increased on the skin it produces gelling, providing an effect of reducing or eliminating the effect of migration of the composition outside the application area. As a result of investigation by the present inventors, however, it has been found that the reduction of fluidity at high temperature is sometimes insufficient with the composition disclosed in the aforementioned publication, while the long-term storage stability is also inadequate.

So it is an object of the invention to provide an oil-in-water-type emulsion cosmetic that can maintain ultraviolet shielding ability even when at high temperature after application, and that also has excellent long-term storage stability.

### SUMMARY OF THE INVENTION

The present invention provides an oil-in-water-type emulsion cosmetic comprising (1) water, (2) an organic UV filter, (3) a thermo-thickening polymer and (4) an anionic surfactant, wherein
(3) the thermo-thickening polymer is
(3a) a cellulosic thermo-thickening polymer containing a cyclodextrin and an alkyl cellulose represented by the following formula (i):

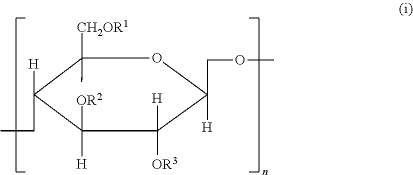

[where R¹, R² and R³ each independently represent a hydrogen atom, an alkyl group having 1 to 6 carbon atoms, —[CH₂CH₂₋ₖ(CH₃)ₖO]ₘH or —CH₂CH(OH)CH₂OCⱼH₂ⱼ₊₁, n represents a number of 100 to 10,000, k represents 0 or 1, m represents a number of 1 to 10 and j represents a number of 6 to 26, with the proviso that R¹, R² and R³ are not all hydrogen atoms], or
(3b) a triblock polyoxyalkylene-based thermo-thickening polymer with a lower critical solution temperature, in which a triblock structure composed of polyethylene oxide-polypropylene oxide-polyethylene oxide is linked via at least one from among urethane bond, urea bond, allophanate bond and biuret bond,
and wherein in a case where (3a) cellulosic thermo-thickening polymer is used as (3) the thermo-thickening polymer, (4) the anionic surfactant is at least one selected from the group consisting of (4a) an anionic surfactant having the following chemical formula (ii) and (4b) an anionic surfactant having the following chemical formula (iii),
in a case where (3b) cellulosic thermo-thickening polymer is used as (3) the thermo-thickening polymer, (4) the anionic surfactant is at least one selected from the group consisting of (4a) an anionic surfactant having the following chemical formula (ii), (4b) an anionic surfactant having the following chemical formula (iii) and (4c) an anionic surfactant having the following chemical formula (iv)

CₚH₂ₚ₊₁—OPO(OH)OM (ii)

Cₚ₋₁H₂ₚ₋₁—CONH—R¹⁰—(COOM)_{q} (iii)

Cₚ₋₁H₂ₚ₋₁—COO—CH₂CHOH—CH₂—OCO—R²⁰—(COOM)ᵣ (iv)

[where p is a number of 12 to 22, q is 1 or 2, r is a number of 1 to 3, R¹⁰ is a (q+1)-valent organic group, R²⁰ is a (r+1)-valent organic group and M is a hydrogen atom or a monovalent metal.]

In a particular embodiment, the water-miscible volatile organic solvent content of the oil-in-water-type emulsion cosmetic is less than 1 mass %. In another particular embodiment, a latex film former is not included in the composition of the invention.

The “oil-in-water emulsified cosmetic” may also be referred to hereunder as “oil-in-water emulsion” or “oil-in-water emulsion composition” or “cosmetic composition in form of and oil-in-water emulsion”. The “mass %” may also be referred to hereunder as “% by weight”.

The oil-in-water-type emulsion cosmetic is not only able to maintain its ultraviolet shielding ability (for example, Sun Protection Factor, SPF) even when at high temperature (for example, 40° C.) after application, but can also increase the ultraviolet shielding ability. The long-term storage stability (for example, storage for 3 months at 45° C.) is also excellent.

In other words, it is possible to provide a method for maintaining and increasing the Sun Protection Factor of an oil-in-water-type emulsion cosmetic that has been applied onto skin, under conditions of higher temperature than at the time of application, using an oil-in-water-type emulsion cosmetic of the invention as the oil-in-water-type emulsion cosmetic.

There is further provided a cosmetic process for caring for keratinic substances, and particularly skin, onto which the oil-in-water-type emulsion cosmetic of the present invention has been applied. The thermo-thickening polymers in prior documents easily lose thermal stability in the oil-in-water-type emulsion cosmetic. On the other hand, according to the present invention, the thermo-thickening polymers are stabilized because such polymers are combined with specific surfactant(s).

### Advantageous Effects of Invention

According to the invention it is possible to provide an oil-in-water-type emulsion cosmetic that can maintain ultraviolet shielding ability even when at high temperature after application, and that also has excellent long-term storage stability.

### DETAILED DESCRIPTION OF THE INVENTION

Embodiments of the invention will now be described. However, the present invention is not limited to the embodiments described below.

### Aqueous Media

The oil-in-water-type emulsion cosmetic is a cosmetic comprising (1) aqueous medium comprising water. The aqueous medium may consist of water alone, or it may also include a solvent that is soluble in water. Water may be distilled water, purified water, hot spring water, deep water, or plant-derived steam distilled water such as lavender water, rose water or orange flower water. The content of water may be 20 to 80 mass %, 30 to 70 mass % or 45 to 60 mass %, based on the total mass of the oil-in-water-type emulsion cosmetic.

Examples of solvents that are soluble in water include mono-alcohols such as methanol, ethanol, propyl alcohol, isopropyl alcohol and phenoxyethanol; and polyhydric alcohols such as ethylene glycol, 1,3-propanediol, propylene glycol, butylene glycol, pentylene glycol, hexylene glycol, dipropylene glycol, polyethylene glycol, glycerin, diglycerin, polyglycerin, sorbitol, xylitol and mannitol, and mixtures thereof. The solvent that is soluble in water may be a single one used alone, or two or more may be used in combination.

The oil-in-water-type emulsion cosmetic comprises a UV filter as component (2), and comprises either (2a) an organic UV filter, (2b) an inorganic UV filter, and mixtures thereof. In a particular embodiment the oil-in-water-type emulsion cosmetic comprises at least an organic UV filter. It is preferred that the oil-in-water-type emulsion cosmetic contains (2a) an organic UV filter only, or it contains (2a) an organic UV filter and (2b) an inorganic UV filter.

### Organic UV filters

An organic UV filter is largely classified into (2a-L) a lipophilic organic UV filter and (2a-H) a hydrophilic organic UV filter.

(2a-L) a lipophilic organic UV filter is further classified as (2a-L1) a lipophilic organic UV-A filter, (2a-L2) a lipophilic organic UV-B filter and (2a-L3) a lipophilic UV-A and UV-B filter, while (2a-H) hydrophilic organic UV filter is further classified as (2a-H1) a hydrophilic organic UV-A filter, (2a-H2) a hydrophilic organic UV-B filter and (2a-H3) a hydrophilic organic UV-A and UV-B filter

### Lipophilic Organic Fillers

The term “lipophilic organic UV filter” means organic UV filter which is soluble in oils at a concentration of at least 1% by weight relative to the total weight of the oil at room temperature (25° C.) and atmosphere pressure (10⁵ Pa).

In a particular embodiment, the oil-in-water-type emulsion cosmetic comprises at least a lipophilic organic filter selected in the group consisting of aminobenzophenone compounds, dibenzoylmethane compounds, anthranilic acid compounds, 4,4-diaryl butadiene compounds, cinnamate compounds, salicylic compounds, anilino triazine compounds, benzoic acid compounds, diphenylacrylate compounds, benzylidene camphor compounds, phenylbenzimidazole compounds, imidazoline compounds, benzalmalonate compounds and mecocyanine compounds, and mixtures thereof.

In a particular embodiment, the oil-in-water-type emulsion cosmetic comprises at least a lipophilic organic filter selected in the group consisting of aminobenzophenone compounds, cinnamate compounds, salicylic compounds, anilino triazine , bis-resorcinyl triazine compounds, and mixtures thereof.

(2a-L1) lipophilic organic UV-A filter include aminobenzophenone compounds, dibenzoylmethane compounds, anthranilic acid compounds and 4,4-diaryl butadiene compounds.

Aminobenzophenone compounds include diethylamino hydroxybenzoyl hexyl benzoate (DHHB, product name: Uvinul A+ by BASF). Dibenzoylmethane compounds include 4-isopropyldibenzoylmethane (product name: Eusolex 8020 by Merck), 1-(4-methoxy-1-benzofuran-5-yl)-3-phenylpropane-1,3-dione (product name: Pongamol by Quest), 1-(4-(tert-butyl)phenyl)-3-(2-hydroxyphenyl)propane-1,3-dione and butyl methoxydibenzoylmethane (product name: Parsol 1789 by DSM).

Anthranilic acid compounds include menthyl anthranilate (product name: NEO HELIPAN MA by Symrise). 4,4-diarylbutadiene compounds include 1,1-dicarboxy(2,2′-dimethylpropyl)-4,4-diphenylbutadiene, diphenyl butadiene malonate and malononitrile.

(2a-L2) Lipophilic organic UV-B V filters include cinnamate compounds, salicylic compounds, anilino triazine compounds, benzoic acid compounds, diphenylacrylate compounds, benzylidene camphor compounds, phenylbenzimidazole compounds, imidazoline compounds, benzalmalonate compounds and mecocyanine compounds.

Cinnamate compounds include ethylhexyl methoxycinnamate (product name: PARSOL CX by DSM), isopropyl ethoxycinnamate, isoamyl methoxycinnamate (product name: NEO HELIOPAN E 1000 by Symrise), diisopropyl methylcinnamate, cinoxate, glyceryl ethylhexanoate dimethoxycinnamate, octyl cinnamate, isopropylethyl cinnamate, diisopropylethyl cinnamate, methyldiisopropyl cinnamate, propylmethoxy cinnamate, isopropylmethoxy cinnamate, isoamylmethoxy cinnamate, octylmethoxy cinnamate, cyclohexylmethoxy cinnamate, ethyl-α-cyano-β-phenyl cinnamate, 2-ethylhexyl-α-cyano-β-phenyl cinnamate, glyceryl mono-2-ethylhexanoyl-diparamethoxy cinnamate and 3,4,5-trimethoxy-3-methyl-4-cinnamate[methylbis(trimethylsiloxy)silyl]butyl.

Salicylic compounds include ethylhexyl salicylate (product name: NEO HELIOPAN OS by Symrise), homosalate (product name: Eusolex HMS by Rona/EM industries), amyl salicylate, menthyl salicylate, homomenthyl salicylate, octyl salicylate, phenyl salicylate, benzyl salicylate and p-isopropanolphenyl salicylate.

Anilino triazine compounds include tris-anilino triazine compounds such as ethylhexyl triazone (product name: UVINUL T-150 by BASF), 2,4,6-tris(dineopentyl 4′-aminobenzalmalonate)-s-triazine and 2,4,6-tris(diisobutyl 4′-aminobenzalmalonate)-s-triazine, and bis-anilino triazine compounds such as diethylhexyl butamido triazone (product name: UVASORB HEB by SIGMA 2 V).

Benzoic acid compounds include para-aminobenzoates (PABA) such as ethyl PABA (para-aminobenzoate), ethyl dihydroxypropyl PABA, ethylhexyl dimethyl PABA, PABA monoglycerin ester, N,N-dipropoxy PABA ethyl ester, N,N-diethoxy PABA ethyl ester, N,N-dimethyl PABA ethyl ester and N,N-dimethyl PABA butyl ester.

Diphenylacrylate compounds include octocrylene (product name: UVINUL N539 by BASF) and etocrylene (product name: UVINUL N35 by BASF). Benzylidenecamphor compounds include 3-benzylidene camphor (product name: MEXORYL SD by CHIMEX), methylbenzylidene camphor (product name: EUSOLEX 6300 by MERCK), polyacrylamidomethyl benzylidene camphor (product name: MEXORYL SW by CHIMEX), and terephthalylidene dicamphor sulfonic acid (product name: Mexoryl SX by Chimex).

Phenylbenzimidazole compounds include phenylbenzimidazole sulfonic acid (product name: Eusolex 232 by Merck). Imidazoline compounds include ethylhexyl dimethoxybenzylidene dioxoimidazoline propionate. Benzalmalonate compounds include polyorganosiloxanes containing a benzalmalonate moiety, such as Polysilicone-15 (product name: Parsol SLX by DSM) and di-neopentyl 4′-methoxybenzalmalonate, for example.

(2a-L3) lipophilic UV-A and UV-B filters include benzophenone compounds, such as benzophenone-1 (product name: UVINUL 400 by BASF), benzophenone-2 (product name: UVINUL 500 by BASF), benzophenone-3 (product name: UVINUL M40 by BASF), benzophenone-6 (product name: Helisorb 11 by Norquay), benzophenone-8 (product name: Spectra-Sorb UV-24 by American Cyanamid), benzophenone-10, benzophenone-11 and benzophenone-12; benzotriazole compounds such as drometrizole trisiloxane (product name: Silatrizole by Rhodia Chimie) and bumetrizole (product name: TINOGUARTD AS by CIBA); bis-resorcinyl triazine compounds such as bis-ethylhexyloxyphenol methoxyphenyl triazine (product name: TINOSORB S by CIBA); and benzoxazole compounds such as 2,4-bis[5-(1-dimethylpropyl)benzoxazol-2-yl(4-phenyl)imino]-6-(2-ethylhexyl)imino-1,3,5-triazine (product name: Uvasorb K2A by Sigma 3 V).

### Hydrophilic Organic UV Fillers

(2a) an organic UV filter may include (2a-H) a hydrophilic organic UV filter. The term “hydrophilic organic UV filter” means an organic UV filter which is soluble in water at a concentration of at least 1% by weight relative to the total weight of the water at room temperature (25° C.) and atmosphere pressure (10⁵ Pa). A water dispersion of (2a-L) a lipophilic organic UV filter in water (or water and polyol(s)) can be also regarded as (2a-H) a hydrophilic organic UV filter.

In a particular embodiment, the oil-in-water-type emulsion cosmetic comprises at least a hydrophilic organic filter selected from the group consisting of terephthalylidene dicamphor sulfonic acid and salts thereof, bisbenzoxazolyl derivatives, benzimidazole sulfonic acid compounds and salts thereof, p-aminobenzoic (PABA) derivatives and salts thereof, salicylic acids and salts thereof, DEA methoxycinnamate and salts thereof, benzylidene camphor sulfonic acid and salts thereof, camphor benzalkonium methosulfate and salts thereof, benzophenone-4 and salts thereof, and mixtures thereof.

(2a-H1) Hydrophilic organic UV-A filters include terephthalylidene dicamphor sulfonic acid and salts thereof (product name: Mexoryl SX by Chimex), bisbenzoxazolyl derivatives, such as disodium phenyl dibenzimidazole tetrasulfonate, and salts thereof (product name: Neo Heliopan AP by Haarmann and Reimer, for example).

(2a-H2) Hydrophilic organic UV-B filters include p-aminobenzoic (PABA) derivatives, such as PABA, glyceryl PABA and PEG-25 PABA, and salts thereof (product name: Uvinul P25 by BASF, for example), benzimidazole sulfonic acid compounds such as phenylbenzimidazole sulfonic acid and salts thereof (product name: Eusolex 232 by Merck, for example), ferulic acid and salts thereof, salicylic acids and salts thereof, DEA methoxycinnamate and salts thereof, benzylidene camphor sulfonic acid and salts thereof (product name: Mexoryl SL by Chimex, for example), and camphor benzalkonium methosulfate and salts thereof (product name: Mexoryl SO by Chimex, for example).

An example of a water dispersion of (2a-L) a lipophilic organic UV filter is a water dispersion of (2a-L3) a lipophilic UV-A and UV-B filter. Component (2a-L3) includes a water dispersion such as a water dispersion of an alkylene bis-benzotriazolylphenol compound such as methylene bis-benzotriazolyl tetramethylbutylphenol (product name: TINOSORB M by BASF).

(2a-H3) Hydrophilic organic UV-A and UV-B filters include benzophenone-4 and salts thereof (product name: Uvinul MS40 by BASF, for example), benzophenone-5 and salts thereof, and benzophenone-9 and salts thereof.

(2a) an organic UV filter preferably includes (2a-L) a lipophilic organic UV filter and (2a-H) a hydrophilic organic UV filter component. In this case, (2a-L) lipophilic organic UV filter preferably comprises component (2a-L1), component (2a-L2) and component (2a-L3), where component (2a-L1) is an aminobenzophenone compound, component (2a-L2) is a combination of a cinnamate compound, a salicylic compound and an anilino triazine compound, and component (2a-L3) is a bis-resorcinyl triazine compound. (2a-H) hydrophilic organic UV filter, on the other hand, preferably comprises component (2a-H2) and component (2a-H3), where component (2a-H2) is a benzimidazole sulfonic acid compound and component (2a-H3) is a water dispersion of an alkylene bis-benzotriazolylphenol compound.

Component (2) may comprise (2b) an inorganic UV filter. The inorganic UV filter may be in the form of fine particles with a mean primary particle diameter in the range of 1 nm to 50 μm, preferably 5 nm to 500 nm and more preferably 10 nm to 200 nm. The mean primary particle diameter is based on the arithmetic mean diameter.

The oil-in-water-type emulsion cosmetic may further comprise an (2b) inorganic UV filter, that is preferably a hydrophilic-treated or untreated metal oxide. Such metal oxides include titanium oxide and zinc oxide.

The total content of (2) UV filter (s) and in particular of organic UV filter may range from 2 to 50 mass %, 5 to 40 mass % or 15 to 25 mass %, based on the total mass of the oil-in-water-type emulsion cosmetic.

### Thermo-Thickening Polymer

(3) the thermo-thickening polymer in the oil-in-water-type emulsion cosmetic is selected from (3a) a cellulosic thermo-thickening polymer and (3b) a triblock polyoxyalkylene-based thermo-thickening polymer. The term “thermo-thickening polymer” means a polymer wherein the molecules become structured by heating, forming a network shape, and resulting in increased viscosity and/or progressive gelation. (3) the thermo-thickening polymer is soluble and/or swellable in water.

### Cellulosic Thermo-Thickening Polymer

(3a) the cellulosic thermo-thickening polymer contains a cyclodextrin and an alkyl cellulose represented by the following formula (i). In the formula, R¹, R² and R³ each independently represent a hydrogen atom, an alkyl group having 1 to 6 carbon atoms, —[CH₂CH₂₋ₖ(CH₃)ₖO]ₘH or —CH₂CH(OH)CH₂OCⱼH₂ⱼ₊₁, n represents a number of 100 to 10,000, k represents 0 or 1, m represents a number of 1 to 10 and j represents a number of 6 to 26. This is with the proviso that R¹, R² and R³ are not all hydrogen atoms.

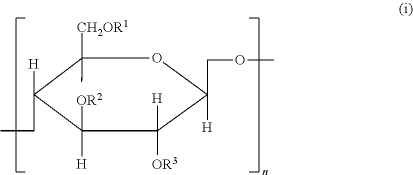

The term “cyclodextrin” means a cyclodextrin or a cyclodextrin derivative. A cyclodextrin is an α-1,4-bonded cyclic oligosaccharide composed of glucopyranose units. An α-cyclodextrin is a cyclic oligosaccharide composed of 6 glucose units. A β-cyclodextrin is a cyclic oligosaccharide composed of 7 glucose units. A γ-cyclodextrin is a cyclic oligosaccharide composed of 8 glucose units. The α-cyclodextrin, β-cyclodextrin and γ-cyclodextrin can be obtained by enzymatic treatment of starch to produce non-reducing cyclic dextrin.

Cyclodextrin include α-cyclodextrin, β-cyclodextrin, γ-cyclodextrin, methylated α-cyclodextrin, methylated β-cyclodextrin, methylated γ-cyclodextrin, dimethyl-α-cyclodextrin, dimethyl-β-cyclodextrin, dimethyl-γ-cyclodextrin, hydroxypropyl-α-cyclodextrin, hydroxyethyl-β-cyclodextrin, hydroxypropyl-β-cyclodextrin, hydroxypropyl-γ-cyclodextrin, hydroxybutyl-β-cyclodextrin, 2-hydroxypropyl-β-cyclodextrin, 2-hydroxybutyl-β-cyclodextrin, 2-hydroxypropyl-α-cyclodextrin and 2-hydroxypropyl-γ-cyclodextrin. A preferred cyclodextrin is γ-cyclodextrin, because it has excellent thermo-thickening properties.

The term “cyclodextrin derivative” means a compound wherein some or all of the hydrogens of the hydroxyl groups at the 2-position, 3-position or 6-position in the glucose molecule composing the cyclodextrin have been substituted with other functional groups.

The other functional groups may be alkyl groups having 1 to 4 carbon atoms and hydroxyalkyl groups having 1 to 4 carbon atoms. Alkyl groups having 1 to 4 carbon atoms are preferably one or more alkyl groups selected from the group consisting of methyl, ethyl, n-propyl, isopropyl, n-butyl, isobutyl, sec-butyl and t-butyl groups. Hydroxyalkyl groups having 1 to 4 carbon atoms are preferably one or more hydroxyalkyl groups selected from the group consisting of hydroxymethyl, 2-hydroxyethyl, 2-hydroxypropyl, 2,3 -dihydroxypropyl, 2-hydroxybutyl, 2-hydroxyisobutyl, dihydroxymethyl and 2,2-dihydroxyethyl groups.

A cyclodextrin derivative is preferably a cyclodextrin wherein some or all of the hydrogens of the hydroxyl groups at the 2-position, 3-position or 6-position in the glucose molecule composing the cyclodextrin have been substituted with one or more groups selected from among alkyl groups having 1 to 2 carbon atoms and hydroxyalkyl groups having 1 to 4 carbon atoms.

Preferred as cyclodextrin derivatives having one or more alkyl groups having 1 to 4 carbon atoms are methylated α-cyclodextrin, methylated β-cyclodextrin, methylated γ-cyclodextrin, dimethyl-α-cyclodextrin, dimethyl-β-cyclodextrin and dimethyl-γ-cyclodextrin. Preferred as cyclodextrin derivatives having one or more hydroxyalkyl groups having 1 to 4 carbon atoms are hydroxypropyl-α-cyclodextrin, hydroxyethyl-β-cyclodextrin, hydroxypropyl-β-cyclodextrin, hydroxypropyl-γ-cyclodextrin and hydroxybutyl-β-cyclodextrin.

The cyclodextrin and cyclodextrin derivative may form a salt thereof, and an example of a salt includes a sodium salt, a potassium salt and a magnesium salt.

An alkyl cellulose represented by formula (i) composing (3a) cellulosic thermo-thickening polymer preferably includes at least one —CH₂CH(OH)CH₂OCⱼH₂ⱼ₊₁ as R¹, R² and R³, with —CⱼH₂ⱼ₊₁ preferably being stearyl group (—C₁₈H₃₇). Alkyl groups having 1 to 6 carbon atoms as R¹, R² and R³ are preferably alkyl groups having 1 to 3 carbon atoms, with methyl and ethyl groups being particularly preferred. The group “—[CH₂CH₂₋ₖ(CH₃)ₖO]ₘH” as R¹, R² or R³ means −[CH₂CH₂O]ₘH or —[CH₂CH(CH₃)O]ₘH. R¹, R² and R³ may be either one or both of these groups, but they preferably include at least —[CH₂CH (CH₃)O]ₘH.

An alkyl cellulose represented by formula (i) preferably includes an alkyl group having 1 to 6 carbon atoms at 10.0 to 50.0 mass %, and more preferably at 21.5 to 30.0 mass %. An alkyl cellulose represented by formula (i) also preferably includes —[CH₂CH₂₋ₖ(CH₃)ₖO]ₘH at 3.0 to 20.0 mass %, and more preferably at 7.0 to 11.0 mass %. In addition, an alkyl cellulose represented by formula (i) preferably includes —CH₂CH(OH)CH₂OCⱼH₂ⱼ₊₁ at 0.1 to 10.0 mass %, more preferably at 0.1 to 2.0 mass % and most preferably at 0.1 to 1.0 mass %.

In the cellulosic thermo-thickening polymer, a cyclodextrin may be combined at 0.01 to 1 part by mass (preferably 0.01 to 0.5 part by mass), with respect to 100 parts by mass of the alkyl cellulose represented by formula (i).

The cellulosic thermo-thickening polymer (3a) used may be the one sold under the trade name Celgela (such as Celgela TS9) by Daido Chemical Corporation.

### Triblock Polyoxyalkylene-Based Thermo-Thickening Polymer

(3b) a triblock polyoxyalkylene-based thermo-thickening polymer may also be used as component (3). (3b) the triblock polyoxyalkylene-based thermo-thickening polymer is a polymer with a lower critical solution temperature (LCST), wherein a triblock structure consisting of polyethylene oxide (PEO)-polypropylene oxide (PPO)-polyethylene oxide (PEO) is linked via at least one from among urethane bond, urea bond, allophanate bond and biuret bond.

The triblock structure consisting of PEO-PPO-PEO may be represented by formula (I) below, for example. In the formula, x represents a number of 20 to 120, y represents a number of 20 to 120, z represents a number of 20 to 120 and m represents a number of 1 or greater.

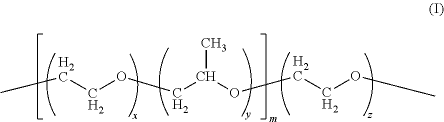

(3b) the triblock polyoxyalkylene-based thermo-thickening polymer can be obtained by reacting a compound having hydroxyl groups as both ends of formula (I), with a polyisocyanate (particularly a diisocyanate). The type of catalyst may be selected for the reaction to introduce urethane bonds, allophanate bonds or biuret bonds, allowing the triblock structure consisting of PEO-PPO-PEO to be linked by the bonds. A urea bond can be formed, for example, by reacting a polyisocyanate (particularly a diisocyanate) having a triblock structure consisting of PEO-PPO-PEO, with a polyamine (particularly a diamine).

At below the LCST, (3b) the triblock polyoxyalkylene-based thermo-thickening polymer dissolves in water as the bonds between the hydrophilic portions (such as PEO portions) and water are strengthened, while at the LCST and above, the hydrophobic bonds of the hydrophobic portions (such as PPO portions) within the polymer molecules and between the polymer molecules are strengthened, resulting in association and aggregation of the polymer chains, increased micelle sizes and higher viscosity.

(3b) the triblock polyoxyalkylene-based thermo-thickening polymer used may be the one sold under the trade name ExpertGel EG312 (exhibiting viscosity increase at 26° C. and high viscosity at 46° C.) and/or ExpertGel EG412 (exhibiting viscosity increase at 20° C. and high viscosity at 34° C.), by DKSH North America.

The total content of (3) thermo-thickening polymer may range from 0.05 to 4 mass %, 0.1 to 3 mass % or 0.1 to 2 mass %, based on the total mass of the oil-in-water-type emulsion cosmetic.

### Anionic Surfactant

The oil-in-water-type emulsion cosmetic contains (4) an anionic surfactant, but when it contains (3a) cellulosic thermo-thickening polymer as (3) thermo-thickening polymer, then (4) anionic surfactant is at least one selected from the group consisting of (4a) an alkyl phosphoric acid ester having 12 to 22 carbon atoms, (4b) an amide of alkylcarboxylic acid having 12 to 22 carbon atoms and an amino acid, a salt thereof, and mixtures thereof.

In a particular embodiment, when oil-in-water-type emulsion cosmetic contains (3b) the triblock polyoxyalkylene-based thermo-thickening polymer as (3) thermo-thickening polymer, then (4) anionic surfactant is at least one selected from the group consisting of (4a) an alkyl phosphoric acid ester having 12 to 22 carbon atoms, (4b) an amide of alkylcarboxylic acid having 12 to 22 carbon atoms and an amino acid, and (4c) a monoester of a polycarboxylic acid and a glyceride of an alkylcarboxylic acid having 12 to 22 carbon atoms, a salt thereof, and mixtures thereif.

(4a) the alkyl phosphoric acid ester having 12 to 22 carbon atoms may be represented by formula (ii)

CₚH₂ₚ₊₁—OPO(OH)OM (ii)

wherein p is a number of 12 to 22, and M is a hydrogen atom or a monovalent metal.

When M is a monovalent metal, component (4a) will be a salt. Sodium and potassium are preferred as monovalent metals.

(4a) the alkyl phosphoric acid ester having 12 to 22 carbon atoms is preferably an alkyl phosphoric acid ester having 12 to 20 carbon atoms or a salt thereof, and more preferably an alkyl phosphoric acid ester having 14 to 18 carbon atoms or a salt thereof. That is, in formula (ii), p is preferably 12 to 20 and more preferably 14 to 18.

(4a) the alkyl phosphoric acid ester having 12 to 22 carbon atoms may be dodecylphosphoric acid, tridecylphosphoric acid, tetradecylphosphoric acid, pentadecylphosphoric acid, hexadecylphosphoric acid (cetylphosphoric acid), heptadecylphosphoric acid, octadecylphosphoric acid, nonadecylphosphoric acid, eicosylphosphoric acid, heneicosylphosphoric acid, docosylphosphoric acid, or a salt (such as a sodium salt or potassium salt) thereof.

(4b) the amide of alkylcarboxylic acid having 12 to 22 carbon atoms and an amino acid may be represented by formula (iii) below

Cₚ₋₁H₂ₚ₋₁—CONH—R¹⁰—(COOM)_{q} (iii)

wherein, R¹⁰ is a (q+1)-valent organic group, p is a number of 12 to 22, q is 1 or 2, and M is a hydrogen atom or a monovalent metal.

When M is a monovalent metal, component (4b) will be a salt. Sodium and potassium are preferred as monovalent metals.

(4b) the amide of alkylcarboxylic acid having 12 to 22 carbon atoms and an amino acid is preferably an amide of an alkylcarboxylic acid having 14 to 22 carbon atoms and an amino acid, or a salt (such as a sodium salt or potassium salt) thereof, and more preferably an amide of an alkylcarboxylic acid having 16 to 20 carbon atoms and an amino acid, or a salt (such as a sodium salt or potassium salt) thereof. That is, in formula (iii), p is preferably 14 to 22 and more preferably 16 to 20. Examples of alkylcarboxylic acid are myristic acid, palmitic acid and stearic acid.

In formula (iii), the —NH—R¹⁰—(COOM)_{q} portion is an amino acid residue. The amino acid of (4b) amide of alkylcarboxylic acid having 12 to 22 carbon atoms and an amino acid is preferably an amino acid having one amino group and one carboxy group, or an amino acid having one amino group and two carboxy groups.

Amino acids having one amino group and one carboxy group include glycine, alanine, valine, leucine, isoleucine, serine, threonine, proline, asparagine, glutamine, cysteine, methionine, phenylalanine and tyrosine. Amino acids having one amino group and two carboxy groups include aspartic acid and glutamic acid. Preferred amino acids are those having one amino group and two carboxy groups, with glutamic acid being especially preferred. (4b) the amide of alkylcarboxylic acid having 12 to 22 carbon atoms and an amino acid may form a salt such as a sodium salt or potassium salt, and when the amino acid is one with two carboxy groups, at least one of them may form a salt.

(4c) the monoester of a polycarboxylic acid and a glyceride of an alkylcarboxylic acid having 12 to 22 carbon atoms may be represented by formula (iv)

Cₚ₋₁H₂ₚ₋₁—COO—CH₂CHOH—CH₂—OCO—R²⁰—(COOM)ᵣ (iv)

wherein R²⁰ is a (q+1)-valent organic group, p is a number of 12 to 22, r is a number of 1 to 3, and M is a hydrogen atom or a monovalent metal.

When M is a monovalent metal, component (4c) will be a salt. Sodium and potassium are preferred as monovalent metals.

(4c) the monoester of a polycarboxylic acid and a glyceride of an alkylcarboxylic acid having 12 to 22 carbon atoms is preferably a monoester of a polycarboxylic acid and a glyceride of an alkylcarboxylic acid having 14 to 22 carbon atoms, or a salt thereof, and more preferably a monoester of a polycarboxylic acid and a glyceride of an alkylcarboxylic acid having 16 to 20 carbon atoms, or a salt thereof. That is, in formula (iv), p is preferably 14 to 22 and more preferably 16 to 20.

In formula (iv), the CO—R²⁰—(COOM)ᵣ portion is a polycarboxylic acid residue. The polycarboxylic acid of component (4c) may be a dicarboxylic acid, tricarboxylic acid or tetracarboxylic acid. The polycarboxylic acid is preferably a tricarboxylic acid, in which case r in formula (iv) is 2.

Dicarboxylic acids include oxalic acid, malonic acid, succinic acid, glutaric acid, adipic acid, pimelic acid, suberic acid, azelaic acid, sebacic acid, phthalic acid, isophthalic acid and terephthalic acid. Tricarboxylic acids include citric acid, trimellitic acid and aconitic acid. Tetracarboxylic acids include 1,2,3,4-butanetetracarboxylic acid, pyromellitic acid and 3,3′,4,4′-biphenyltenacarboxylic acid.

(4c) the monoester of a polycarboxylic acid and a glyceride of an alkylcarboxylic acid having 12 to 22 carbon atoms is preferably a monoester of citric acid and a glyceride of an alkylcarboxylic acid of 18 carbon atoms (stearic acid), or a salt (such as a sodium salt or potassium salt) thereof.

The total content of anionic surfactants (4a), (4b) and (4c) may range from 0.05 to 4 mass %, 0.1 to 3 mass % or 0.1 to 2 mass %, based on the total mass of the oil-in-water-type emulsion cosmetic.

The total contents of (1) an aqueous medium, (2) an organic UV filter, (3) thermo-thickening polymer and (4) an anionic surfactant in the oil-in-water-type emulsion cosmetic range most preferably from 45 to 60 mass %, 15 to 25 mass %, 0.1 to 2 mass % or 0.1 to 2 mass %, based on the total mass of each.

The total content of (3a) cellulosic thermo-thickening polymer can range from 0.2 to 1.6 mass % based on the total mass. In this case, the total content of anionic surfactant components (4a), (4b) and (4c) can be 0.4 to 2 mass % based on the total mass. The total content of (3b) triblock polyoxyalkylene-based thermo-thickening polymer can be 0.4 to 2 mass % based on the total mass. In this case, the total content of anionic surfactants components (4a), (4b) and (4c) can be 0.4 to 2 mass % based on the total mass. The ratio of the total content of (3a) cellulosic thermo-thickening polymer with respect to the total content of anionic surfactants components (4a), (4b) and (4c) is preferably 0.1 to 4 and more preferably 0.2 to 2.5 based on mass, and the ratio of the total content of (3b) triblock polyoxyalkylene-based thermo-thickening polymer with respect to the total content of anionic surfactants components (4a), (4b) and (4c) is preferably 0.2 to 5 and more preferably 0.5 to 5 based on mass.

In a particular embodiment, the oil-in-water-type emulsion cosmetic preferably has a water-miscible volatile organic solvent content of less than 1 mass %. The water-miscible volatile organic solvent content is preferably no greater than 0.5 mass % and even more preferably no greater than 0.3 mass %. The water-miscible volatile organic solvent may be a lower alcohol of 1 to 5 carbon atoms (such as methanol, ethanol, propanol, isopropanol, n-butanol, isobutanol or t-butanol). In another particular embodiment, the oil-in-water-type emulsion cosmetic preferably does not contain a latex film former. A latex film former is a substance (such as an emulsion type polymer substance for mascara) that is able to form a film at room temperature (25° C.).

### Additional Ingredients

The oil-in-water-type emulsion cosmetic may also contain components other than component (1) to component (4). For example, it may include an anionic surfactant other than component (4a), (4b) and (4c) as component (4), or it may contain a nonionic surfactant.

Examples include humectants (including polyols such as glycerin, butylene glycol, propanediol and pentylene glycol), thickeners, oils (hydrocarbon oils, fats and oils, waxes, hydrogenated oils, ester oils, fatty acids, silicone oils, fluorine-based oils and the like. Examples are Caprylyl Methicone, Dicaprylyl Carbonate and Butylene Glycol Dicaprylate/Dicaprate.), neutralizers, (such as tromethamine and sodium hydroxide), preservatives, chelating agents (such as EDTA disodium salt) and adsorbents (such as silica).

The viscosity of the oil-in-water-type emulsion cosmetic at 25° C. may be, for example, 50 mPas or higher, 60 mPa·s or higher or 80 mPa·s or higher, to no higher than 10,000 mPa·s, no higher than 5,000 mPa·s or no higher than 3,000 mPa·s. The viscosity of the oil-in-water-type emulsion cosmetic can be measured using a rotating viscometer (for example, Rheolab QC by Anton Paar, spindle: CC-27, rotational speed; 200 rpm, ST-22-4 V-40, rotational speed: 100 rpm, ST-24-2D/2 V/2 V-30, rotational speed: 50 rpm).

The oil-in-water-type emulsion cosmetic described above may be suitably used as a sunscreen. Such composition provides a SPF protection that remains efficient even at high temperature in summer.

The present invention also relates to a cosmetic process for caring for keratinic substances, comprising the application of the oil-in-water-type emulsion cosmetic according to the invention onto keratinic substances, and particularly skin. By keratinic substances, it means healthy keratinic substances, meaning keratinic substances of healthy subjects, e.g not affected by a pathology.

### EXAMPLES

The invention will now be illustrated by examples, with the understanding that the invention is not meant to be limited to these examples.

### Examples 1 to 9, Comparative Examples 1 to 5

Methods for producing oil-in-water-type emulsion cosmetics for the Examples and Comparative Examples will now be described, with the component amounts listed in Tables 1 to 3. The ingredients listed in the compositions are expressed in mass weight % by total mass weight of the composition.

First, the component listed in “B” in Tables 1 to 3 was weighed out into a tank (capacity: 1000 mL). The tank was heated to 80 to 85° C., and homogeneity inside the tank was confirmed. The components listed as “C” and “D” in Tables 1 to 3 were then gradually added to the tank, and after stirring for 20 minutes, the component listed as “A” in Tables 1 to 3 was gradually added to the tank and stirring was continued.

Air-cooling of the tank was then initiated, confirming that the liquid temperature had fallen to below 50° C., and the component listed as “E” in Tables 1 to 3 was added to the tank, stirring was continued, and when it was confirmed that the liquid temperature had fallen to below 40° C., the remaining component (the component listed as “F” in Tables 1 to 3) was added and stirring was continued until the liquid temperature reached 30° C., to obtain an oil-in-water-type emulsion cosmetic.

### Stability

Each of the prepared oil-in-water-type emulsion cosmetics of the Examples and Comparative Examples was filled into a transparent container and sealed with a cap, and then stored for 3 months at 45° C. After storage the phase homogeneity was observed, and an evaluation of ‘OK’ was assigned for those with no change such as separation or an evaluation of ‘NG’ was assigned for those that had exudation of the aqueous layer resulting in loss of phase homogeneity, as shown in Tables 1 to 3.

### Sun Protection Factor

An in vitro measurement was conducted to measure the thermal responsive SPF-boosting effects at ordinary temperature (25° C.) and 40° C. First, following the protocol of ISO24443 (Determination of sunscreen UVA photoprotection in vitro), the oil-in-water-type emulsion cosmetics of the Examples and Comparative Examples were coated onto measurement boards (PMMA plate: HELIOPLATE HD6 by Helioscreen labs) that had been stored at room temperature for 30 minutes, and SPF measurement was conducted at room temperature by using in vitro SPF analyzer: Labsphere UV-20005 transmittance analyzer by Helioscreen labs. The measurement board that had been stored for half a day in a thermostatic bath set to 40° C. was removed from the thermostatic bath, and the oil-in-water-type emulsion cosmetic of the Example or Comparative Example was coated onto the measurement board. It was then returned to the 40° C. thermostatic bath and allowed to stand for 30 minutes for stabilization, after which it was removed from the thermostatic bath and SPF measurement was immediately conducted in the same manner as above, at room temperature (25° C.). The results are shown in Tables 1, 2 and 3.

The results presented in the Tables 1 -3 show that the combination of an organic UV filter, a particular thermo-thickening polymer and an anionic surfactant, provide an oil-in-water type emulsion with excellent stability improved. SPF value at 40° C., in comparison with other comparative examples without thermos-thickening polymers.

TABLE 1

| | | | INCI Name | Exam- | Exam- | Exam- | Exam- | Exam- |
|---|---|---|---|---|---|---|---|---|
| | Component | | (Product Name) | ple 1 | ple 2 | ple 3 | ple 4 | ple 5 |
| A | (2) | UV filter | ETHYLHEXYL METHOXYCINNAMATE | 2.98 | 2.98 | 2.98 | 2.98 | 2.98 |
| A | (2) | UV filter | ETHYLHEXYL SALICYLATE | 0.75 | 0.75 | 0.75 | 0.75 | 0.75 |
| A | (2) | UV filter | ETHYLHEXYL TRIAZONE | 1.00 | 1.00 | 1.00 | 1.00 | — |
| A | (2) | UV filter | BIS-ETHYLHEXYLOXYPHENOL METHOXYPHENYL | 2.00 | 2.00 | 2.00 | 2.00 | 1.00 |
| | | | TRIAZINE | | | | | |
| A | (2) | UV filter | ETHYLHEXYL METHOXYCINNAMATE, | 5.80 | 5.80 | 5.80 | 5.80 | 5.80 |
| | | | DIETHYLAMINO HYDROXYBENZOYL HEXYL | | | | | |
| | | | BENZOATE | | | | | |
| A | | Oil | CAPRYLYL METHICONE | 2.00 | 2.00 | 2.00 | 2.00 | 2.00 |
| A | | Oil | DICAPRYLYL CARBONATE | 4.00 | 4.00 | 4.00 | 4.00 | 4.00 |
| A | | Oil | BUTYLENE GLYCOL DICAPRYLATE/DICAPRATE | 10.00 | 10.00 | 10.00 | 10.00 | 10.00 |
| A | | Nonionic emulsifier | POLYGLYCERYL-6 STEARATE and | 1.50 | 1.50 | 1.50 | 1.50 | 1.50 |
| | | | POLYGLYCERYL-6 BEHENATE | | | | | |
| A | | Nonionic emulsifier | C14-22 ALCOHOL AND C12-20 ALKYL GLUCOSDE | — | — | — | — | — |
| B | (1) | Water | Water | 43.95 | 50.07 | 47.87 | 53.37 | 55.37 |
| B | (4a) | Anionic emulsifier | POTASSIUM CETYL PHOSPHATE (Product Name: | 0.60 | — | — | — | — |
| | | | AMPHISOL K) | | | | | |
| B | (4b) | Anionic emulsifier | SODIUM STEAROYL GLUTAMATE (Product | — | 0.20 | 0.20 | 0.20 | 0.20 |
| | | | Name. AMISOFT HS-11 PF MB) | | | | | |
| B | (4c) | Anionic emulsifier | GLYCERYL STEARATE CITRATE (Product | — | — | — | — | — |
| | | | Name: DERMOFEEL GSC INF 100 CFU/G) | | | | | |
| B | | Anionic emulsifier | SODIUM DILAURAMIDOGLUTAMIDE LYSINE | — | — | — | — | — |
| B | | Preservative | CHLORPHENESIN | 0.27 | — | — | — | — |
| B | | Preservative | PHENOXYETHANOL | — | 0.80 | 0.80 | 0.80 | 0.80 |
| B | | Chelate agent | DISODIUM EDTA | 0.20 | 0.20 | 0.20 | 0.20 | 0.20 |
| C | (3a) | Thermo-thickening | HYDROXYPROPYLMETH- | 0.30 | 0.30 | 0.30 | 0.30 | 0.30 |
| | | polymer | YL CELLULOSE STEAROXYETHER, | | | | | |
| | | | CYCLODEXTRIN (Product Name: CELGELA TS9) | | | | | |
| C | (3b) | Thermo-thickening | POLOXAMER 338, PPG-12/SMDI COPOLYMER (Product | — | — | — | — | — |
| | | polymer | Name: EXPERTGEL EG312) | | | | | |
| C | (3b) | Thermo-thickening | POLOXAMER 407, PPG-12/SMDI COPOLYMER (Product | — | — | — | — | — |
| | | polymer | Name: EXPERTGEL EG412) | | | | | |
| C | | Thickener | AGAR | — | — | — | — | — |
| C | | Thickener | AMMONIUM ACRYLOYLDIMETHYL TAURATE/ | 0.75 | — | — | — | — |
| | | | VP COPOLYMER (Product Name: | | | | | |
| | | | ARISTOFLEX AVC) | | | | | |
| C | | Thickener | AMMONIUM ACRYLOYLDIMETHYL TAURATE/ | — | 0.50 | 0.50 | 0.50 | 0.50 |
| | | | BEHENETH-25 METHACRYLATE WATER andT BUTYL | | | | | |
| | | | ALCOHOL (Product Name: ARISTOFLEX HMB) | | | | | |
| D | | Thickener | SODIUM HYDROXYETHYL | 0.30 | 0.30 | — | — | — |
| | | | ACRYLATE/ACRYLOYLDIMETHYL | | | | | |
| | | | TAURATE COPOLYMER, SQUALANE, POLYSORBATE | | | | | |
| | | | 60 and WATER (Product Name: SIMULGEL | | | | | |
| | | | NS MB) | | | | | |
| D | | Thickener | ACRYLATES COPOLYMER and WATER (Product Name: | — | — | — | — | — |
| | | | CARBOPOL AQUA SF-1) | | | | | |
| E | (2) | UV filter | PHENYLBENZIMIDAZOLE SULFONIC ACID | 1.00 | 1.00 | 1.00 | 1.00 | 1.00 |
| E | | Neutralizer | TROMETHAMINE | 0.75 | 0.75 | 0.75 | 0.75 | 0.75 |
| E | | Neutralizer | SODIUM HYDROXIDE | — | — | — | — | — |
| F | | Polyol | BUTYLENE GLYCOL | 3.00 | 3.00 | 3.00 | 3.00 | 3.00 |
| F | | Polyol | PROPANEDIOL | 6.00 | 6.00 | 5.00 | 5.00 | 5.00 |
| F | | Polyol | PENTYLENE GLYCOL | 3.00 | 2.00 | — | — | — |
| F | (2) | UV filter | METHYLENE BIS-BENZOTRIAZOLYL | 4.50 | 4.50 | 10.00 | 4.50 | 4.50 |
| | | | TETRAMETHYLBUTYLPHENOL, | | | | | |
| | | | WATER, DECYL GLUCOSIDE, PROPYLENE | | | | | |
| | | | GLYCOL and XANTHAN GUM | | | | | |
| F | | Adsorbent | SILICA | 0.35 | 0.35 | 0.35 | 0.35 | 0.35 |
| | | | Total | 100.00 | 100.00 | 100.00 | 100.00 | 100.00 |

| | | | | | |
|---|---|---|---|---|---|
| Stability (45 C., 3 months) | Stable | Stable | Stable | Stable | Stable |
| Evaluation of Stability | OK | OK | OK | OK | OK |
| in vitro SPF at 25 C. | 50 | 54 | 69 | 46 | 24 |
| in vitro SPF at 40 C. | 67 | 66 | 137 | 60 | 57 |

TABLE 2

| | | | INCI Name | Exam- | Exam- | Exam- | Exam- |
|---|---|---|---|---|---|---|---|
| | Component | | (Product Name) | ple 6 | ple 7 | ple 8 | ple 9 |
| A | (2) | UV filter | ETHYLHEXYL METHOXYCINNAMATE | 2.98 | 2.98 | 2.98 | 2.98 |
| A | (2) | UV filter | ETHYLHEXYL SALICYLATE | 0.75 | 0.75 | 0.75 | 0.75 |
| A | (2) | UV filter | ETHYLHEXYL TRIAZONE | 1.00 | 1.00 | 1.00 | 1.00 |
| A | (2) | UV filter | BIS-ETHYLHEXYLOXYPHENOL METHOXYPHENYL TRIAZINE | 2.00 | 2.00 | 2.00 | 2.00 |
| A | (2) | UV filter | ETHYLHEXYL METHOXYCINNAMATE, | 5.80 | 5.80 | 5.80 | 5.80 |
| | | | DIETHYLAMINO HYDROXYBENZOYL HEXYL BENZOATE | | | | |
| A | | Oil | CAPRYLYL METHICONE | 2.00 | 2.00 | 2.00 | 2.00 |
| A | | Oil | DICAPRYLYL CARBONATE | 4.00 | 4.00 | 4.00 | 4.00 |
| A | | Oil | BUTYLENE GLYCOL DICAPRYLATE/DICAPRATE | 10.00 | 10.00 | 10.00 | 10.00 |
| A | | Nonionic emulsifier | POLYGLYCERYL-6 STEARATE and POLYGLYCERYL-6 BEHENATE | — | 1.50 | — | — |
| A | | Nonionic emulsifier | C14-22 ALCOHOL AND C12-20 ALKYL GLUCOSIDE | 1.50 | — | 1.50 | 1.50 |
| B | (1) | Water | Water | 57.67 | 57.87 | 52.07 | 52.07 |
| B | (4a) | Anionic emulsifier | POTASSIUM CETYL PHOSPHATE (Product Name: AMPHISOL K) | — | 0.50 | — | — |
| B | (4b) | Anionic emulsifier | SODIUM STEAROYL GLUTAMATE (Product Name: AMISOFT | 0.20 | — | 0.20 | — |
| | | | HS-11 PF MB) | | | | |
| B | (4c) | Anionic emulsifier | GLYCERYL STEARATE CITRATE (Product Name: DERMOFEEL | — | — | — | 0.20 |
| | | | GSC INF 100 CFU/G) | | | | |
| B | | Anionic emulsifier | SODIUM DILAURAMIDOGLUTAMIDE LYSINE | — | — | — | — |
| B | | Preservative | CHLORPHENESIN | — | — | — | — |
| B | | Preservative | PHENOXYETHANOL | 0.80 | 0.80 | 0.90 | 0.90 |
| B | | Chelate agent | DISODIUM EDTA | 0.20 | 0.20 | 0.20 | 0.20 |
| C | (3a) | Thermo-thickening | HYDROXYPROPYL METHYLCELLULOSE STEAROXYETHER, | — | — | — | — |
| | | polymer | CYCLODEXTRIN (Product Name: CELGELA TS9) | | | | |
| C | (3b) | Thermo-thickening | POLOXAMER 338, PPG-12/SMDI COPOLYMER (Product | — | 0.50 | 0.50 | 0.50 |
| | | polymer | Name: EXPERTGEL EG312) | | | | |
| C | (3b) | Thermo-thickening | POLOXAMER 407, PPG-12/SMDI COPOLYMER (Product | 1.00 | — | — | — |
| | | polymer | Name: EXPERTGEL EG412) | | | | |
| C | | Thickener | AGAR | — | — | — | — |
| C | | Thickener | AMMONIUM ACRYLOYL DIMETHYL TAURATE/VP COPOLYMER | — | — | — | — |
| | | | (Product Name: ARISTOFLEX AVC) | | | | |
| C | | Thickener | AMMONIUM ACRYLOYLDIMETHYL TAURATE/BEHENETH-25 | 0.50 | 0.50 | 0.50 | 0.50 |
| | | | METHACRYLATE, WATER andT BUTYL ALCOHOL (Product | | | | |
| | | | Name: ARISTOFLEX HMB) | | | | |
| D | | Thickener | SODIUM HYDROXYETHYL ACRYLATE/ | — | — | — | — |
| | | | ACRYLOYL DIMETHYL TAURATE COPOLYMER, | | | | |
| | | | SQUALANE, POLYSORBATE 60 and | | | | |
| | | | WATER (Product Name: SIMULGEL NS MB) | | | | |
| D | | Thickener | ACRYLATES COPOLYMER and WATER (Product Name: | 1.00 | 1.00 | 1.50 | 1.50 |
| | | | CARBOPOL AQUA SF-1) | | | | |
| E | (2) | UV filter | PHENYLBENZIMIDAZOLE SULFONIC ACID | 1.00 | 1.00 | 1.00 | 1.00 |
| E | | Neutralizer | TROMETHAMINE | — | — | — | — |
| E | | Neutralizer | SODIUM HYDROXIDE | 0.25 | 0.25 | 0.25 | 0.25 |
| F | | Polyol | BUTYLENE GLYCOL | 3.00 | 3.00 | 3.00 | 3.00 |
| F | | Polyol | PROPANEDIOL | — | — | 5.00 | 5.00 |
| F | | Polyol | PENTYLENE GLYCOL | — | — | — | — |
| F | (2) | UV filter | METHYLENE BIS-BENZOTRIAZOLYL TETRAMETH- | 4.00 | 4.00 | 4.50 | 4.50 |
| | | | YLBUTYLPHENOL, WATER, DECYL GLUCOSIDE | | | | |
| | | | PROPYLENE GLYCOL and XANTHAN GUM | | | | |
| F | | Adsorbent | SILICA | 0.35 | 0.35 | 0.35 | 0.35 |
| | | | Total | 100.00 | 100.00 | 100.00 | 100.00 |

| | | | | |
|---|---|---|---|---|
| Stability (45 C., 3 months) | Stable | Stable | Stable | Stable |
| Evaluation of Stability | OK | OK | OK | OK |
| in vitro SPF at 25 C. | 71 | 49 | 55 | 38 |
| in vitro SPF at 40 C. | 79 | 90 | 65 | 97 |

TABLE 3

| | | | | Compar- | Compar- | Compar- | Compar- | Compar- |
|---|---|---|---|---|---|---|---|---|
| | | | INCI Name | ative | ative | ative | ative | ative |
| | Component | | (Product Name) | Example 1 | Example 2 | Example 3 | Example 4 | Example 5 |
| A | (2) | UV filter | ETHYLHEXYL METHOXYCINNAMATE | 2.98 | 2.98 | 2.98 | 2.98 | 2.98 |
| A | (2) | UV filter | ETHYLHEXYL SALICYLATE | 0.75 | 0.75 | 0.75 | 0.75 | 0.75 |
| A | (2) | UV filter | ETHYLHEXYL TRIAZONE | 1.00 | 1.00 | 1.00 | 1.00 | 1.00 |
| A | (2) | UV filter | BIS-ETHYLHEXYLOXYPHENOL | 2.00 | 2.00 | 2.00 | 2.00 | 2.00 |
| | | | METHOXYPHENYL TRIAZINE | | | | | |
| A | (2) | UV filter | ETHYLHEXYL METHOXYCINNAMATE, | 5.80 | 5.80 | 5.80 | 5.80 | 5.80 |
| | | | DIETHYLAMINO HYDROXYBENZOYL HEXYL | | | | | |
| | | | BENZOATE | | | | | |
| A | | Oil | CAPRYLYL METHICONE | 2.00 | 2.00 | 2.00 | 2.00 | 2.00 |
| A | | Oil | DICAPRYLYL CARBONATE | 4.00 | 4.00 | 4.00 | 4.00 | 4.00 |
| A | | Oil | BUTYLENE GLYCOL DICAPRYLATE/DICAPRATE | 10.00 | 10.00 | 10.00 | 10.00 | 10.00 |
| A | | Nonionic | POLYGLYCERYL-6 STEARATE and | 1.50 | 1.50 | 1.50 | — | — |
| | | emulsifier | POLYGLYCERYL-6 BEHENATE | | | | | |
| A | | Nonionic | C14-22 ALCOHOL AND C12-20 ALKYL | — | — | — | 1.50 | 1.50 |
| | | emulsifier | GLUCOSIDE | | | | | |
| B | (1) | Water | Water | 50.07 | 49.67 | 57.89 | 52.57 | 52.07 |
| B | (4a) | Anionic | POTASSIUM CETYL PHOSPHATE (Product | — | 0.60 | 0.50 | — | — |
| | | emulsifier | Name: AMPHISOL K) | | | | | |
| B | (4b) | Anionic | SODIUM STEAROYL GLUTAMATE (Product | — | — | — | 0.20 | — |
| | | emulsifier | Name: AMISOFT HS-11 PF MB) | | | | | |
| B | (4c) | Anionic | GLYCERYL STEARATE CITRATE (Product | 0.20 | — | — | — | — |
| | | emulsifier | Name: DERMOFEEL GSC INF 100 CFU/G) | | | | | |
| B | | Anionic | SODIUM DILAURAMIDOGLUTAMIDE LYSINE | — | — | — | — | 0.20 |
| | | emulsifier | | | | | | |
| B | | Preservative | CHLORPHENESIN | — | — | — | — | — |
| B | | Preservative | PHENOXYETHANOL | 0.80 | 0.80 | 0.80 | 0.90 | 0.90 |
| B | | Chelate agent | DISODIUM EDTA | 0.20 | 0.20 | 0.20 | 0.20 | 0.20 |
| C | (3a) | Thermo- | HYDROXYPROPYL METHYLCELLULOSE | 0.30 | — | — | — | — |
| | | thickening | STEAROXYETHER, CYCLODEXTRIN (Product | | | | | |
| | | polymer | Name: CELGELA TS9) | | | | | |
| C | (3b) | Thermo- | POLOXAMER 338, PPG-12/SMDI COPOLYMER | — | — | — | — | 0.50 |
| | | thickening | (Product Name: EXPERTGEL EG312) | | | | | |
| | | polymer | | | | | | |
| C | (3b) | Thermo- | POLOXAMER 407, PPG-12/SMDI COPOLYMER | — | — | — | — | — |
| | | thickening | (Product Name: EXPERTGEL EG412) | | | | | |
| | | polymer | | | | | | |
| C | | Thickener | AGAR | — | 0.30 | — | — | — |
| C | | Thickener | AMMONIUM ACRYLOYLDIMETHYL TAURATE/VP | — | — | — | — | — |
| | | | COPOLYMER (Product Name: ARISTOFLEX AVC) | | | | | |
| C | | Thickener | AMMONIUM ACRYLOYLDIMETHYL TAURATE/ | 0.50 | 0.50 | 0.50 | 0.50 | 0.50 |
| | | | BEHENETH-25 METHACRYLATE, WATER andT | | | | | |
| | | | BUTYL ALCOHOL (Product Name: ARISTOFLEX | | | | | |
| | | | HMB) | | | | | |
| D | | Thickener | SODIUM HYDROXYETHYL ACRYLATE/ACRYLOYL | 0.30 | 0.30 | — | — | — |
| | | | DIMETHYL TAURATE COPOLYMER, SQUALANE, | | | | | |
| | | | POLYSORBATE 60 and WATER (Product | | | | | |
| | | | Name: SIMULGEL NS MB) | | | | | |
| D | | Thickener | ACRYLATES COPOLYMER and WATER (Product | — | — | 1.00 | 1.50 | 1.50 |
| | | | Name: CARBOPOL AQUA SF-1) | | | | | |
| E | (2) | UV filter | PHENYLBENZIMIDAZOLE SULFONIC ACID | 1.00 | 1.00 | 1.00 | 1.00 | 1.00 |
| E | | Neutralizer | TROMETHAMINE | 0.75 | 0.75 | — | — | — |
| E | | Neutralizer | SODIUM HYDROXIDE | — | — | 0.23 | 0.25 | 0.25 |
| F | | Polyol | BUTYLENE GLYCOL | 3.00 | 3.00 | 3.00 | 3.00 | 3.00 |
| F | | Polyol | PROPANEDIOL | 6.00 | 6.00 | — | 5.00 | 5.00 |
| F | | Polyol | PENTYLENE GLYCOL | 2.00 | 2.00 | — | — | — |
| F | (2) | UV filter | METHYLENE BIS-BENZOTRIAZOLYL TETRAMETH- | 4.50 | 4.50 | 4.50 | 4.50 | 4.50 |
| | | | YLBUTYLPHENOL, WATER, DECYL | | | | | |
| | | | GLUCOSIDE, PROPYLENE GLYCOL and | | | | | |
| | | | XANTHAN GUM | | | | | |
| F | | Adsorbent | SILICA | 0.35 | 0.35 | 0.35 | 0.35 | 0.35 |
| | | | Total | 100.00 | 100.00 | 100.00 | 100.00 | 100.00 |

| | | | | | |
|---|---|---|---|---|---|
| Stability (45 C., 3 months) | Aqueous | Aqueous | Aqueous | Stable | Aqueous |
| | exudate | exudate | exudate | | exudate |
| Evaluation of Stability | NG | NG | NG | OK | NG |
| in vitro SPF at 25 C. | — | 29 | — | 49 | — |
| in vitro SPF at 40 C. | — | 28 | — | 34 | — |

1-16. (canceled)

17. An oil-in-water-type emulsion cosmetic comprising (1) an aqueous medium, (2) an organic UV filter, (3) a thermo-thickening polymer, and (4) an anionic surfactant, wherein
(3) the thermo-thickening polymer is
(3a) a cellulosic thermo-thickening polymer containing a cyclodextrin and an alkyl cellulose represented by the following formula (i): 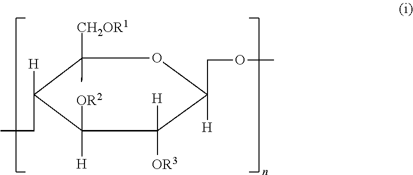
where R¹, R² and R³ each independently represent a hydrogen atom, an alkyl group having 1 to 6 carbon atoms, —[CH₂CH₂₋ₖ(CH₃)ₖO]ₘH or —CH₂CH(OH)CH₂OCⱼH₂ⱼ₊₁, n represents a number of 100 to 10,000, k represents 0 or 1, m represents a number of 1 to 10 and j represents a number of 6 to 26, with the proviso that R¹, R² and R³ are not all hydrogen atoms, or
(3b) a triblock polyoxyalkylene-based thermo-thickening polymer with a lower critical solution temperature, in which a triblock structure composed of polyethylene oxide-polypropylene oxide-polyethylene oxide is linked via at least one from among urethane bond, urea bond, allophanate bond and biuret bond,
and wherein in a case where (3a) cellulosic thermo-thickening polymer is used as (3) the thermo-thickening polymer, (4) the anionic surfactant is at least one selected from the group consisting of (4a) an anionic surfactant having the following chemical formula (ii) and (4b) an anionic surfactant having the following chemical formula (iii),
in a case where (3b) cellulosic thermo-thickening polymer is used as (3) the thermo-thickening polymer, (4) the anionic surfactant is at least one selected from the group consisting of (4a) an anionic surfactant having the following chemical formula (ii), (4b) an anionic surfactant having the following chemical formula (iii) and (4c) an anionic surfactant having the following chemical formula (iv).
CₚH₂ₚ₊₁—OPO(OH)OM (ii)
Cₚ₋₁H₂ₚ₋₁—CONH—R¹⁰—(COOM)_{q} (iii)
Cₚ₋₁H₂ₚ₋₁—COO—CH₂CHOH—CH₂—OCO—R²⁰—(COOM)ᵣ (iv)
where p is a number of 12 to 22, q is 1 or 2, r is a number of 1 to 3, R¹⁰ is a (q+1)-valent organic group, R²⁰ is a (r+1)-valent organic group and M is a hydrogen atom or a monovalent metal.

18. The oil-in-water-type emulsion cosmetic according to claim 17, wherein component (4a) is cetylphosphoric acid or a salt thereof, component (4b) is stearoylglutamic acid or a salt thereof, and component (4c) is glyceryl stearate citrate or a salt thereof.

19. The oil-in-water-type emulsion cosmetic according to claim 17, wherein: component (2a) comprises
(2a-L) a lipophilic organic UV filter which is at least one type selected from the group consisting of (2a-L1) a lipophilic organic UV-A filter, (2a-L2) a lipophilic organic UV-B filter and (2a-L3) a lipophilic UV-A and UV-B filter, and
(2a-H) a hydrophilic organic UV filter which is at least one type selected from the group consisting of (2a-H1) a hydrophilic organic UV-A filter, (2a-H2) a hydrophilic organic UV-B filter and (2a-H3) a hydrophilic organic UV-A and UV-B filter.

20. The oil-in-water-type emulsion cosmetic according to claim 19, wherein
component (2a-L) comprises component (2a-L1), component (2a-L2) and component (2a-L3),
component (2a-L1) being an aminobenzophenone compound,
component (2a-L2) being a combination of a cinnamate compound, a salicylic compound and an anilino triazine compound, and
component (2a-L3) being a bis-resorcinyl triazine compound.

21. The oil-in-water-type emulsion cosmetic according to claim 19, wherein
component (2a-H) comprises component (2a-H2) and component (2a-H3),
component (2a-H2) being a benzimidazole sulfonic acid compound and
component (2a-H3) being a water dispersion of an alkylene bis-benzotriazolylphenol compound.

22. The oil-in-water-type emulsion cosmetic according to claim 17, wherein component (2) contains (2b) an inorganic UV filter,
(2b) the inorganic UV filter being a hydrophilic-treated or untreated metal oxide.

23. The oil-in-water-type emulsion cosmetic according to claim 17, wherein the content of component (1) is 20 to 80 mass % based on the total mass.

24. The oil-in-water-type emulsion cosmetic according to claim 17, wherein the total content of component (2) is 2 to 50 mass % based on the total mass.

25. The oil-in-water-type emulsion cosmetic according to claim 17, wherein the total content of component (3) is 0.05 to 4 mass % based on the total mass.

26. The oil-in-water-type emulsion cosmetic according to claim 17, wherein the total content of components (4a), (4b) and (4c) is 0.05 to 4 mass % based on the total mass.

27. The oil-in-water-type emulsion cosmetic according to claim 17, wherein the contents of component (1), the total content of component (2), the total content of component (3), and the total content of components (4a), (4b), and (4c) are respectively 45 to 60 mass %, 15 to 25 mass %, 0.1 to 2 mass %, or 0.1 to 2 mass %, based on the total mass.

28. The oil-in-water-type emulsion cosmetic according to claim 17, wherein the ratio of the total content of component (3a) with respect to the total content of components (4a), (4b), and (4c) is 0.1 to 4, based on mass.

29. The oil-in-water-type emulsion cosmetic according to claim 17, wherein the ratio of the total content of component (3b) with respect to the total content of components (4a), (4b), and (4c) is 0.2 to 5, based on mass.

30. The oil-in-water-type emulsion cosmetic according to claim 17, wherein the cosmetic is a sunscreen.

31. A method for maintaining or increasing the Sun Protection Factor of an oil-in-water-type emulsion cosmetic that has been applied onto skin, under conditions of higher temperature than at the time of application,
wherein the oil-in-water-type emulsion cosmetic according to claim 17 is used as the oil-in-water-type emulsion cosmetic.

32. A cosmetic process for caring for keratinic substances, comprising the application of the oil-in-water-type emulsion cosmetic according to claim 17 onto keratinic substances.
